SEQUENCE LISTING (9 sequences)

SEQ ID NO: 1 - protein, 120 aa, synthetic construct
EVQLQESGPGLVKPSETLSLTCAVSGYSITSGYYWNWIRQPPGKKLEWMGYINYGGSNGYNPSLKSRITISRDTSKNQFSLKLSSVTAADTAVYYCARDYDAYYEALDDWGQGTTVTVSS

SEQ ID NO: 2 - protein, 106 aa, synthetic construct
EIVLSQSPATLSLSPGERATLSCRASSRVNYMHWYQQKPGQSPRPWISATSNLASGVPARFSGSGSGTSYTLTISSLEPEDFAVYYCQQWSSNPPTFGGGTKVEIK

SEQ ID NO: 3 - protein, 6 aa, synthetic construct
SGYYWN

SEQ ID NO: 4 - protein, 16 aa, synthetic construct
YINYGGSNGYNPSLKS

SEQ ID NO: 5 - protein, 11 aa, synthetic construct
DYDAYYEALDD

SEQ ID NO: 6 - protein, 10 aa, synthetic construct
RASSRVNYMH

SEQ ID NO: 7 - protein, 7 aa, synthetic construct
ATSNLAS

SEQ ID NO: 8 - protein, 9 aa, synthetic construct
QQWSSNPPT

SEQ ID NO: 9 - protein, 574 aa, synthetic construct
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

(19) United States
(12) Patent Application Publication
(10) Pub. No.: US 2025/0352645 A1
(43) Pub. Date: Nov. 20, 2025

(54) BIOMARKERS AND METHODS FOR TREATING NSCLC

(71) Applicants: I-MAB BIOPHARMA CO., LTD., Shanghai (CN); SHANGHAI JUNSHI BIOSCIENCES CO., LTD., Shanghai (CN)

(72) Inventors: Yanni ZHANG, Shanghai (CN); Yuan MENG, Shanghai (CN); Zhengyi WANG, Shanghai (CN); Xuejun LIU, Rockville, MD (US); Yanling NIU, Shanghai (CN)

(21) Appl. No.: 18/869,686

(22) PCT Filed: May 26, 2023
(86) PCT No.: PCT/CN2023/096557

§ 371 Date: Nov. 26, 2024

(30) Foreign Application Priority Data
May 26, 2022 (WO) PCT/CN2022/095171

(51) Int. Cl. A61K 39/395; A61K 39/00; C07K 16/28; C07K 16/40; G01N 33/574

(52) U.S. Cl. CPC A61K 39/39558; C07K 16/2818; C07K 16/2896; C07K 16/40; C12Y 301/03005; G01N 33/57492; A61K 2039/507; A61K 2039/545; G01N 2333/70596; G01N 2333/916

(57) ABSTRACT

Biomarkers and methods for the treatment of non-small cell lung cancer (NSCLC). Biomarkers for patient selection and prognosis, and diagnostic kit for analyzing the biomarkers.

### FIELD

The present application relates to biomarkers and methods for the treatment of non-small cell lung cancer (NSCLC). In particular, provided are biomarkers for patient selection and prognosis, and diagnostic kits for analyzing the biomarker.

### BACKGROUND

Non-small-cell lung carcinoma (NSCLC) is any type of epithelial lung cancer other than small-cell lung carcinoma (SCLC). It accounts for about 85% of all lung cancers. Compared to small-cell carcinoma, NSCLC is relatively insensitive to chemotherapy. The survival rates for stages I through IV of NSCLC decrease significantly due to the advancement of the disease. For stage I, the five-year survival rate is 47%, stage II is 30%, stage III is 10%, and stage IV is merely 1%.

The treatment approach for people who has advanced NSCLC is first aimed at relieving pain and distress via chemotherapy such as cisplatin which indiscriminately target all rapidly dividing cells, and via targeted agents which are more tailored to specific genetic aberrations found within the tumor. However, the needs of improved therapeutic approaches exist.

### SUMMARY

The present application addresses the clinical needs.

In one aspect, provided herein is a method for treating a subject with NSCLC, comprising: (a) determining level of CD73 in a sample of the subject, (b) evaluating whether the subject is more likely to respond to or benefit from a combination treatment of a CD73 antagonist and a PD1/PD-L1 antagonist based on the level of CD73, (c) if the subject is more likely to respond to or benefit from the combination treatment, administering the combination treatment to the subject.

In another aspect, provided herein is a method for selecting a therapy for a subject with NSCLC, the method comprising: (a) determining level of CD73 in a sample of the subject, and (b) evaluating whether the subject is more likely to respond to or benefit from a combination treatment of a CD73 antagonist and a PD1/PD-L1 antagonist based on the level of CD73, (c) if the subject is more likely to respond to or benefit from the combination treatment, selecting the combination treatment for the subject.

In another aspect, provided herein is a method for identifying a subject with NSCLC is more likely to respond to or benefit from a combination treatment of a CD73 antagonist and a PD1/PD-L1 antagonist, comprising: (a) determining level of CD73 in a sample of the subject, (b) evaluating whether the subject is more likely to respond to or benefit from the combination treatment based on the level of CD73, and (c) if the subject is more likely to respond to or benefit from the combination treatment, providing a recommendation of the combination treatment to the subject.

In another aspect, provided herein is a method for predicting responsiveness of a subject with NSCLC to a combination treatment of a CD73 antagonist and a PD1/PD-L1 antagonist, comprising: (a) determining level of CD73 in a sample of the subject, (b) evaluating whether the subject is more likely to respond to or benefit from the combination treatment based on the level of CD73, and (c) if the subject is more likely to respond to or benefit from the combination treatment, providing a recommendation of the combination treatment to the subject.

In another aspect, provided herein is a method for identifying a subject with NSCLC is more likely to respond to or benefit from a combination treatment of a CD73 antagonist and a PD1/PD-L1 antagonist, comprising: (a) determining level of CD73 in a sample of the subject, wherein an increase of CD73 in the sample relative to that in a reference sample indicates that the subject is more likely to respond to or benefit from the combination treatment, and (b) providing a recommendation that the subject will be more likely to respond to or benefit from the combination treatment.

In another aspect, provided herein is a method for predicting responsiveness of a subject with NSCLC to a combination treatment of a CD73 antagonist and a PD1/PD-L1 antagonist, comprising: (a) determining level of CD73 in a sample of the subject, wherein an increase of CD73 in the sample relative to that in a reference sample indicates that the subject is more likely to respond to or benefit from the combination treatment, and (b) providing a recommendation that the subject will have an increased likelihood of being responsive to or benefit from the combination treatment.

In another aspect, provided herein is method for treating a subject with non-small cell lung cancer (NSCLC), comprising: (a) identifying a subject of NSCLC as likely to respond to or benefit from a combination treatment of a CD73 antagonist and a PD1/PD-L1 antagonist based on the level of CD73 in sample of the subject, and (b) administering the CD73 antagonist and the PD1/PD-L1 antagonist to the identified subject.

In another aspect, provided herein is a method for selecting a therapy for a subject with NSCLC, the method comprising: (a) determining the level of CD73 in a sample of the subject, and (b) evaluating whether the subject is likely to respond to or benefit from a combination treatment of a CD73 antagonist and a PD1/PD-L1 antagonist based on the level of CD73, (c) selecting the combination treatment for the subject when the subject is determined as likely to respond to or benefit from the combination treatment.

In another aspect, provided herein is a method for treating a subject with NSCLC, comprising administering a CD73 antagonist and a PD1/PD-L1 antagonist to a subject with NSCLC having a level of CD73 in a sample indicating as likely to respond to or benefit from a combination treatment of the CD73 antagonist and the PD1/PD-L1 antagonist.

In another aspect, provided herein is a method for predicting responsiveness of a subject with NSCLC to a combination treatment of a CD73 antagonist and a PD1/PD-L1 antagonist, comprising: (a) determining the level of CD73 in a sample of the subject, (b) evaluating whether the subject is likely to respond to or benefit from the combination treatment based on the level of CD73, and (c) providing a recommendation of the combination treatment to the subject when the subject is determined as likely to respond to or benefit from the combination treatment.

In another aspect, provided herein is a method for identifying a subject with NSCLC as more likely to respond to or benefit from a combination treatment of a CD73 antagonist and a PD1/PD-L1 antagonist than a reference patient, comprising: (a) determining the level of CD73 in a sample of the subject, wherein an increase of the level of CD73 in the sample relative to that in a reference sample from the reference patient indicates that the subject is more likely to respond to or benefit from the combination treatment than the reference patient, and (b) providing a recommendation that the subject will be more likely to respond to or benefit from the combination treatment than the reference patient.

In another aspect, provided herein is a method for predicting responsiveness of a subject with NSCLC to a combination treatment of a CD73 antagonist and a PD1/PD-L1 antagonist, comprising: (a) determining level of CD73 in a sample of the subject, wherein an increase of CD73 in the sample relative to that in a reference sample from a reference patient indicates that the subject is more likely to respond to or benefit from the combination treatment than the reference patient, and (b) providing a recommendation that the subject will have an increased likelihood of being responsive to or benefit from the combination treatment as compared to the reference patient.

In another aspect, provided herein is use of a CD73 antagonist and a PD1/PD-L1 antagonist for the manufacture of a medicament for treating a subject with NSCLC having a level of CD73 in a sample indicating as likely to respond to or benefit from a combination treatment of the CD73 antagonist and the PD1/PD-L1 antagonist.

In another aspect, provided herein is use of a CD73 antagonist for the manufacture of a medicament for treating a subject with NSCLC having a level of CD73 in a sample indicating as likely to respond to or benefit from a combination treatment of the CD73 antagonist and a PD1/PD-L1 antagonist, wherein the subject is also administered the PD1/PD-L1 antagonist.

In another aspect, provided herein is use of a PD1/PD-L1 antagonist for the manufacture of a medicament for treating a subject with NSCLC having a level of CD73 in a sample indicating as likely to respond to or benefit from a combination treatment of a CD73 antagonist and the PD1/PD-L1 antagonist, wherein the subject is also administered the CD73 antagonist.

In some embodiments of the method of the present application, the CD73 antagonist of the combination treatment is a CD73 antibody. In some embodiments, the CD73 antibody is selected from the group consisting of polyclonal antibody, monoclonal antibody, Fab, scFv, diabody, triabody, minibody, VHH and sdAb. In some embodiments, the CD73 antibody comprises: (a) a HCDR1, a HCDR2, and a HCDR3 within a heavy variable region (VH) having the sequence set forth in SEQ ID No: 1, and (b) a LCDR1, a LCDR2, and a LCDR3 within a light variable region (VL) having the sequence set forth in SEQ ID No: 2, wherein the HCDR1, HCDR2, HCDR3, LCDR1, LCDR2, and LCDR3 are according to Kabat numbering scheme.

In some embodiments, the CD73 antibody comprises: (1) a HCDR1 comprising an amino acid sequence as set forth in SEQ ID No. 3, (2) a HCDR2 comprising an amino acid sequence as set forth in SEQ ID No. 4, (3) a HCDR3 comprising an amino acid sequence as set forth in SEQ ID No. 5, (4) a LCDR1 comprising an amino acid sequence as set forth in SEQ ID No. 6, (5) a LCDR2 comprising an amino acid sequence as set forth in SEQ ID No. 7, and (6) a LCDR3 comprising an amino acid sequence as set forth in SEQ ID No. 8.

In some embodiments, the CD73 antibody is selected from the group consisting of uliledlimab (I-Mab Biopharma), oleclumab (AstraZeneca), CPI-006 (Corvus Pharma), BMS-986179 (Bristol-Myers Squibb), AB-680 (Arcus Biosciences), NZV-930 (SRF373, Surface Oncolohgy/Novartis), JAB-BX102 (Jacobio), AK119 (Akesobio), Sym024 (Symphogen), IBI 325 (Innovent), BR 101 (Hisun BioRay), and LY3475070 (Eli-Lilly).

In some embodiments, the PD-1/PD-L1 antagonist of the combination treatment is a PD-1 antibody or a PD-L1 antibody. In some embodiments, the PD-1 antibody is selected from the group consisting of pembrolizumab, nivolumab, toripalimab, pidilizumab, cemiplimab, sintilimab, cetrelimab, spartalizumab, camrelizumab, tislelizumab, balstilimab, dostarlimab, ABBV-181, penpulimab, genolimzumab, retifanlimab, sasanlimab, AMP-224, AB122, F-520, MEDI-3387, MEDI-5771, MEDI-0680, SG-001, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01. In some embodiments, the PD-L1 antibody is selected from the group consisting of atezolizumab, manelimab, avelumab, cosibelimab, durvalumab, envafolimab, socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003.

In some embodiments, the combination treatment further comprises a chemotherapy. In some embodiments, the chemotherapy is selected from the group consisting of platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine, and any combination thereof.

In some embodiments, the combination treatment comprises administrating the CD73 antagonist and the PD-1/PD-L1 antagonist concurrently or separately.

In some embodiments, the NSCLC is a stage III or stage IV NSCLC. In some embodiments, the NSCLC is an advanced NSCLC or a metastatic NSCLC. In some embodiments, the NSCLC is a recurrent NSCLC. In some embodiments, the NSCLC has a squamous histology or a non-squamous histology.

In some embodiments, the subject is treatment naïve. In some embodiments, the treatment is first line treatment. In some embodiments, the subject is ineligible or rejected for the first line treatment. In some embodiments, the first line treatment is selected from the group consisting of chemotherapy, PD-1 antibody therapy, PD-L1 antibody therapy and any combination thereof. In some embodiments, the chemotherapy is selected from the group consisting of platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine, and any combination thereof. In some embodiments, the PD-1 antibody is selected from the group consisting of pembrolizumab, nivolumab, toripalimab, pidilizumab, cemiplimab, sintilimab, cetrelimab, spartalizumab, camrelizumab, tislelizumab, balstilimab, dostarlimab, ABBV-181, penpulimab, genolimzumab, retifanlimab, sasanlimab, AMP-224, AB122, F-520, MEDI-3387, MEDI-5771, MEDI-0680, SG-001, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01. In some embodiments, the PD-L1 antibody is selected from the group consisting of atezolizumab, manelimab, avelumab, cosibelimab, durvalumab, envafolimab, socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003.

In some embodiments, the level of CD73 is protein level of CD73. In some embodiments, the protein level is determined by immunohistochemistry (IHC).

In some embodiments, the sample comprises tumor cells, immune cells such as tumor infiltrating immune cells (TILs), stromal cells and any combinations thereof. In some embodiments, the sample is obtained prior to the combination treatment.

In some embodiments, the level of CD73 comprises a proportion (such as percentage) of CD73-positive cells among all cells in the sample. In some embodiments, the level of CD73 comprises a proportion of CD73-positive tumor cells among all cells in the sample (TC), or a proportion of CD73-positive immune cells among all cells in the sample (IC), whichever is higher (TC/IC). In some embodiments, a CD73 level (TC/IC) of 30% or higher identifies the subject as likely to respond to or benefit from a combination treatment. In some embodiments, a CD73 level (TC/IC) of 35%, 40%, 45% or 50% or higher identifies the subject as likely to respond to or benefit from a combination treatment.

In some embodiments, the level of CD73 comprises a proportion of CD73-positive tumor cells among all cells in the sample (TC(+)), or a proportion of CD73-positive immune cells among all cells in the sample (IC(+)), whichever is higher, wherein the positive is at least moderate positive (TC/IC(+)).

In some embodiments, a CD73 level (TC/IC(+)) of 10% or higher identifies the subject as likely to respond to or benefit from a combination treatment. In some embodiments, a CD73 level (TC/IC(+)) of 15%, 20%, 25%, 30%, 35% or 40% or higher identifies the subject as likely to respond to or benefit from a combination treatment.

In some embodiments, the reference sample comprises a referenced tissue or a reference cell. In some embodiments, reference sample is derived from a healthy subject or a non-diseased sample of the subject.

In some embodiments, the responsiveness comprises a relative increase in one or more of the following: overall survival (OS), progression free survival (PFS), complete response (CR), partial response (PR) and combinations thereof.

In another aspect, provided herein is a kit or an article of manufacture for use in the method of the present application, comprising: 1) one or more reagents for determining level of CD73 in a sample from a subject with NSCLC, and (2) a package insert, wherein the package insert suggests treating the subject with a combination treatment of a CD73 antagonist and a PD1/PD-L1 antagonist based on the level of CD73.

In another aspect, provided herein is a kit or an article of manufacture for use in the method of the present application, comprising: (1) one or more reagents for determining level of CD73 in a sample from a subject with NSCLC, and (2) a package insert, wherein the package insert suggests an increase in the level of CD73 in the sample relative to that in a reference sample indicating the subject is more likely to respond to or benefit from a combination treatment of a CD73 antagonist and a PD1/PD-L1 antagonist.

In some embodiments of the kit or the article of manufacture, the reagent is an anti-CD73 antibody. In some embodiments, the antibody is selected from the group consisting of D7F9A (CST, Cat. No. 13160), 606117 (R&D), EPR6114 (Abcam, Cat. No. ab133582), 4G6E3 (Abcam, Cat. No. ab202122), NT5E/2503 (Abcam, Cat. No. ab257309), NT5E/2505 (Abcam, Cat. No. ab257310), NT5E/2545 (Abcam, Cat. No. ab257311), NT5E/2646 (Abcam, Cat. No. ab257312), 7G2 (Thermo Fisher, Cat. No. 1D7), JM11-40 (Thermo Fisher, Cat. No. JM11-40), CD73 Recombinant Rabbit Monoclonal Antibody (1) (Invitrogen, Cat. No. MA5-29454), BLR054F (Thermo Fisher, Cat. No. BLR054F), sc-32299 (Santa Cruz, Cat. No. IE9), and AD2 (Biorad, Cat. No. AD2).

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

### BRIEF DESCRIPTIONS OF THE DRAWINGS

FIG. 1 illustrates exemplary tumor cells or immune cells with CD73 staining intensities of 0-3.

FIG. 2 illustrates an exemplary section of a NSCLC tumor sample after CD73 staining.

FIGS. 3A and 3B illustrate average CD73 level in subjects with or without prior first line treatment, under different IHC scoring systems.

FIGS. 4A and 4B illustrate correlation of CD73 level with the responses of the subjects to the combination treatment of CD73 antibody and PD-1 antibody, under different IHC scoring systems.

FIGS. 5A and 5B illustrate the correlation of CD73 level with the responses of the subjects to the combination treatment of CD73 antibody and PD-1 antibody, under different IHC scoring systems, analyzed by Pearson Correlation Coefficient.

FIGS. 6A and 6B illustrate preliminary ROC analysis of CD73 level under different IHC scoring systems.

### DETAILED DESCRIPTION

### Definitions

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

As used herein, the term “antibody” is used in the broadest sense and specifically covers intact antibodies (e.g., full length antibodies), antibody fragments (including without limitation Fab, F(ab′)2, scFv, scFv-Fc, single domain antibodies, single heavy chain antibodies, and single light chain antibodies), monoclonal antibodies, and polyclonal antibodies, so long as they exhibit the desired biological activity (e.g., epitope binding).

As used herein, the term “isolated” antibody may refer to an antibody that is substantially free of other cellular material. In one embodiment, an isolated antibody is substantially free of other proteins from the same species. In another embodiment, an isolated antibody is expressed by a cell from a different species and is substantially free of other proteins from the different species. In some embodiments, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. An antibody may be rendered substantially free of naturally associated components (or components associated with the cellular expression system used to produce the antibody) by isolation, using protein purification techniques well known in the art. In some embodiments, the antibody will be purified (1) to greater than 75% by weight of antibody as determined by the Lowry method, and most preferably more than 80%, 90%, 95% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond (also termed a “VH/VL pair”), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. See, e.g., Chothia et al., J. Mol. Biol., 186:651 (1985); Novotny and Haber, Proc. Natl. Acad. Sci. U.S.A., 82:4592 (1985).

As used herein, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. See, e.g., Kabat et al., Sequences of Proteins of Immunological Interest, Fifth Edition, National Institute of Health, Bethesda, Md. (1991). The constant domains are not involved directly in binding an antibody to an antigen, but exhibit various effector functions, such as participation of the antibody in antibody-dependent cellular toxicity. Variable region sequences of interest include the humanized variable region sequences for CD47 antibodies described in detail elsewhere herein.

The term “hypervariable region (HVR)” or “complementarity determining region (CDR)” may refer to the subregions of the VH and VL domains characterized by enhanced sequence variability and/or formation of defined loops. These include three CDRs in the VH domain (H1, H2, and H3) and three CDRs in the VL domain (L1, L2, and L3). H3 is believed to be critical in imparting fine binding specificity, with L3 and H3 showing the highest level of diversity. See Johnson and Wu, in Methods in Molecular Biology 248:1-25 (Lo, ed., Human Press, Totowa, N.J., 2003).

A number of CDR/HVR delineations are known. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs/CDRs are noted below. “Framework” or “FR” residues are those variable domain residues other than the HVR/CDR residues

| Loop | Kabat | AbM | Chothia | Contact | |
|---|---|---|---|---|---|
| L1 | L24-L34 | L24-L34 | L26-L32 | L30-L36 | |
| L2 | L50-L56 | L50-L56 | L50-L52 | L46-L55 | |
| L3 | L89-L97 | L89-L97 | L91-L96 | L89-L96 | |
| H1 | H31-H35B | H26-H35B | H26-H32 | H30-H35B | (Kabat |
| | | | | | Num- |
| | | | | | bering) |
| H1 | H31-H35 | H26-H35 | H26-H32 | H30-H35 | (Chothia |
| | | | | | Num- |
| | | | | | bering) |
| H2 | H50-H65 | H50-H58 | H53-H55 | H47-H58 | |
| H3 | H95-H102 | H95-H102 | H96-H101 | H93-H101 | |

“Extended” HVRs are also known: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH (Kabat numbering).

“Numbering according to Kabat” may refer to the numbering system used for heavy chain variable domains or light chain variable domains of the compilation of antibodies in Kabat et al., supra. The actual linear amino acid sequence may contain fewer or additional amino acids corresponding to a shortening of, or insertion into, a FR or HVR of the variable domain. The Kabat numbering of residues may be determined for a given antibody by alignment at regions of homology of the sequence of the antibody with a “standard” Kabat numbered sequence. Typically, the Kabat numbering is used when referring to a residue in the variable domains (approximately residues 1-107 of the light chain and residues 1-113 of the heavy chain), whereas the EU numbering system or index (e.g., the EU index as in Kabat, numbering according to EU IgG1) is generally used when referring to a residue in the heavy chain constant region.

As used herein, the term “antibody fragment”, and all grammatical variants thereof, are defined as a portion of an intact antibody comprising the antigen binding site or variable region of the intact antibody which, in certain instances, is free of the constant heavy chain domains (i.e. CH2, CH3, and/or CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and or can contain any hinge region sequence found in an intact antibody, and/or can contain a leucine zipper sequence fused to or situated in the hinge region sequence or the constant domain sequence of the heavy chain(s).

The Fab fragment also contains the constant domain of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made in an immortalized B cell or hybridoma thereof, or may be made by recombinant DNA methods.

The monoclonal antibodies herein include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab. F(ab′)₂, and Fv), so long as they exhibit the desired biological activity.

The monoclonal antibodies herein specifically include chimeric antibodies (immunoglobulins) in which a portion of the heavy and or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals. In some embodiments, “treating” a disease such as cancer refers to delaying progression of the disease, i.e., deferring, hindering, slowing, retarding, stabilizing, and or postponing development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “effective amount” is at least the minimum amount required to effect a measurable improvement or prevention of a particular disease (e.g., cancer). An effective amount herein may vary according to factors such as the disease state, age, sex, and weight of the patient, and the ability of a therapeutic agent (or combination of therapeutic agents) to elicit a desired response in the individual. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this disclosure, an effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “subject” for purposes of treatment refers to any animal classified as a mammal, including humans, domestic and farm animals, and zoo, sports, or pet animals, such as dogs.

As used herein, the term “PD-1 binding antagonists” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and or PD-L2. In some embodiments, a PD-1 binding antagonist reduces the negative signal mediated by or through cell surface proteins expressed on T lymphocytes, and other cells, mediated signaling through PD-1 or PD-L1 so as render a dysfunctional T-cell less non-dysfunctional.

As used herein, the term “PD-L1 binding antagonists” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In some embodiments, the PD-L1 binding antagonist inhibits binding of PD-L1 to PD-1 and/or B7-1. In some embodiments, the PD-L1 binding antagonists include PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In some embodiments, a PD-L1 binding antagonist reduces the negative signal mediated by or through cell surface proteins expressed on T lymphocytes, and other cells, mediated signaling through PD-L1 or PD-1 so as render a dysfunctional T-cell less non-dysfunctional.

As used herein, the term “biomarker” refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. Biomarkers include, but are not limited to, polynucleotides, polynucleotide copy number alterations, polypeptides, posttranslational modifications, carbohydrates, and/or glycolipid-based molecules.

The “amount” or “level” of a biomarker associated with an increased clinical benefit to an individual is a detectable level in a biological sample. These can be measured by methods known to one skilled in the art, such as IHC. The level or amount of biomarker assessed can be used to determine the response to the treatment.

As used herein, the terms “level of expression” or “expression level” in general are used interchangeably and generally refer to the amount of a biomarker in a biological sample. “Expression” generally refers to the process by which information (e.g., gene-encoded and or epigenetic) is converted into the structures present and operating in the cell.

“Increased expression.” “increased expression level” or “increased level” refers to an increased expression or increased levels of a biomarker in an individual relative to a reference, such as an individual or individuals who are not suffering from the disease or disorder (e.g., cancer) or an internal control (e.g., housekeeping biomarker).

### CD73

Over the past years, more studies suggest the role of adenosine pathway in tumor progression and metastases. Under tumor environment, increased extracellular adenosine can activate the adenosine receptor subtypes A2A and A2B thereby limits the effector T cell functions, induces immunosuppression, and stimulates angiogenesis.

Extracellular adenosine production is regulated by many enzymes. The classical pathway of extracellular adenosine production is based on sequential reactions mediated by ectonucleotidases. In particular, the extracellular adenosine triphosphate is first hydrolyzed by CD39 into adenosine monophosphate (AMP) and then dephosphorylated into adenosine by CD73. Alternatively, CD38 and CD203a can convert NAD+ into ADP-ribose and ADP-ribose into AMP, respectively. AMP is, in turn, dephosphorylated into adenosine by CD73. In both pathways, CD73 plays key function in extracellular adenosine production.

CD73, cluster of differentiation 73, is also known as 5′-nucleotidase (5′-NT) or ecto-5′-nucleotidase, is an enzyme serves to convert AMP to adenosine. CD73 catalyzes the formation of extracellular adenosine which contributes to the immunosuppressive tumor environment. CD73 exists in two forms: one form is anchored, via glycosylphosphatidylinositol, to the membrane of many cells or extracellular vesicles; the second form is generated upon cleavage from membranes through the action of proteases or phospholipases, and it is found in biological fluids.

### CD73 Antagonist

Preclinical evidence shows that CD73 inhibition prevented adenosine-mediated lymphocyte suppression, increased the activity of CD8+ effector cells, and reduced both MDSCs and Tregs. There are a few anti-CD73 antibodies being developed as potential anticancer agents, but none have been approved for clinical use.

Any suitable CD73 antagonist can be used in the present application. In some embodiments, the CD73 antagonist is a CD73 antibody. Any CD73 antibody known in the art can be used in the present application. In some embodiments, the CD73 antibody is selected from a group consisting of a full-length antibody, Fab, Fab′, F(ab′)2, scFv, and sdAb. In some embodiments, the CD73 antibody is a full-length antibody.

In some embodiments, the CD73 antibody comprises a HCDR1, a HCDR2, and a HCDR3, respectively comprising the amino acid sequences of a CDR1, a CDR2, and a CDR3 within a heavy variable region (VH) as set forth in SEQ ID NO. 1. In some embodiments, the CD73 antibody comprises a HCDR1, a HCDR2, and a HCDR3, respectively comprising the amino acid sequences of a CDR1, a CDR2, and a CDR3 within a heavy variable region (VH) as set forth in SEQ ID NO. 1, wherein the CDR1, a CDR2, and a CDR3 are according to Kabat numbering system. In some embodiments, the CD73 antibody comprises: (1) a HCDR1 comprising an amino acid sequence as set forth in SEQ ID No. 3 or an amino acid sequence with one or more substations as compared to SEQ ID No. 3, (2) a HCDR2 comprising an amino acid sequence as set forth in SEQ ID No. 4 or an amino acid sequence with one or more substations as compared to SEQ ID No. 4, and (3) a HCDR3 comprising an amino acid sequence as set forth in SEQ ID No. 5 or an amino acid sequence with one or more substations as compared to SEQ ID No. 5.

In some embodiments, the CD73 antibody comprises a heavy variable region (VH) comprising an amino acid sequence as set forth in SEQ ID NO. 1 or an amino acid sequence having at least 80%, 85%, 87%, 89%, 90%, 91%, 92%, 93%, 94%, 95%, 96%, 97%, 98%, or 99% identity with SEQ ID NO. 1.

In some embodiments, the CD73 antibody comprises a LCDR1, a LCDR2, and a LCDR3, respectively comprising the amino acid sequences of a CDR1, a CDR2, and a CDR3 within a light variable region (VL) as set forth in SEQ ID NO. 2. In some embodiments, the CD73 antibody comprises a LCDR1, a LCDR2, and a LCDR3, respectively comprising the amino acid sequences of a CDR1, a CDR2, and a CDR3 within a light variable region (VL) as set forth in SEQ ID NO. 2, wherein the CDR1, a CDR2, and a CDR3 are according to Kabat numbering system. In some embodiments, the CD73 antibody comprises: (1) a LCDR1 comprising an amino acid sequence as set forth in SEQ ID No. 6 or an amino acid sequence with one or more substations as compared to SEQ ID No. 6, (2) a LCDR2 comprising an amino acid sequence as set forth in SEQ ID No. 7 or an amino acid sequence with one or more substations as compared to SEQ ID No. 7, and (3) a LCDR3 comprising an amino acid sequence as set forth in SEQ ID No. 8 or an amino acid sequence with one or more substations as compared to SEQ ID No. 8.

In some embodiments, the CD73 antibody comprises a light variable region (VL) comprising an amino acid sequence as set forth in SEQ ID NO. 2 or an amino acid sequence having at least 80%, 85%, 87%, 89%, 90%, 91%, 92%, 93%, 94%, 95%, 96%, 97%, 98%, or 99% identity with SEQ ID NO. 2.

In some embodiments, the CD73 antibody comprises: (1) HCDR1, a HCDR2, and a HCDR3, respectively comprising the amino acid sequences of a CDR1, a CDR2, and a CDR3 within a heavy variable region (VH) as set forth in SEQ ID NO. 1, and (2) LCDR1, a LCDR2, and a LCDR3, respectively comprising the amino acid sequences of a CDR1, a CDR2, and a CDR3 within a light variable region (VL) as set forth in SEQ ID NO. 2. In some embodiments, the CD73 antibody comprises: (1) HCDR1, a HCDR2, and a HCDR3, respectively comprising the amino acid sequences of a CDR1, a CDR2, and a CDR3 within a heavy variable region (VH) as set forth in SEQ ID NO. 1, and (2) LCDR1, a LCDR2, and a LCDR3, respectively comprising the amino acid sequences of a CDR1, a CDR2, and a CDR3 within a light variable region (VL) as set forth in SEQ ID NO. 2 wherein the CDR1, a CDR2, and a CDR3 are according to Kabat numbering system. In some embodiments, the CD73 antibody comprises: (1) a HCDR1 comprising an amino acid sequence as set forth in SEQ ID No. 3 or an amino acid sequence with one or more substations as compared to SEQ ID No. 3, (2) a HCDR2 comprising an amino acid sequence as set forth in SEQ ID No. 4 or an amino acid sequence with one or more substations as compared to SEQ ID No. 4, (3) a HCDR3 comprising an amino acid sequence as set forth in SEQ ID No. 5 or an amino acid sequence with one or more substations as compared to SEQ ID No. 5, (4) a LCDR1 comprising an amino acid sequence as set forth in SEQ ID No. 6 or an amino acid sequence with one or more substations as compared to SEQ ID No. 6, (5) a LCDR2 comprising an amino acid sequence as set forth in SEQ ID No. 7 or an amino acid sequence with one or more substations as compared to SEQ ID No. 7, and (6) a LCDR3 comprising an amino acid sequence as set forth in SEQ ID No. 8 or an amino acid sequence with one or more substations as compared to SEQ ID No. 8.

In some embodiments, the CD73 antibody comprises: (1) a heavy variable region (VH) comprising an amino acid sequence as set forth in SEQ ID NO. 1 or an amino acid sequence having at least 80%, 85%, 87%, 89%, 90%, 91%, 92%, 93%, 94%, 95%, 96%, 97%, 98%, or 99% identity with SEQ ID NO. 1, and (2) a light variable region (VL) comprising an amino acid sequence as set forth in SEQ ID NO. 2 or an amino acid sequence having at least 80%, 85%, 87%, 89%, 90%, 91%, 92%, 93%, 94%, 95%, 96%, 97%, 98%, or 99% identity with SEQ ID NO. 2.

In some embodiments, the CD73 antibody is selected from the group consisting of uliledlimab (I-Mab Biopharma), oleclumab (AstraZeneca), CPI-006 (Corvus Pharma), BMS-986179 (Bristol-Myers Squibb), AB-680 (Arcus Biosciences), NZV-930 (SRF373, Surface Oncolohgy Novartis), JAB-BX102 (Jacobio), AK119 (Akesobio), Sym024 (Symphogen), IBI 325 (Innovent), BR 101 (Hisun BioRay), and LY3475070 (Eli-Lilly). In some embodiments, the CD73 antibody is uliledlimab (I-Mab Biopharma) or oleclumab (AstraZeneca).

### PD-1/PD-L1 Antagonist

Any suitable PD-1/PD-L1 antagonist known in the art can be used in the method of the application. In some embodiments, the PD-1/PD-L1 antagonist includes a small molecule inhibitor of PD-1/PD-L1 signaling. In some embodiments, the PD-1/PD-L1 antagonist includes an antibody or an antigen binding fragment thereof, such as an PD-1 antibody, an PD-L1 antibody or an antigen binding fragment thereof.

In some embodiments, the PD-1/PD-L1 signaling inhibitor is an PD-1 antibody or an antigen binding fragment thereof. Any suitable PD-1 antibody can be used in the present application. Exemplary PD-1 antibody can be used in the method of the present application includes but is not limited to polyclonal antibody, monoclonal antibody, Fab, scFv, diabody, triabody, minibody, VHH and sdAb. Exemplary PD-1 antibody includes but is not limited to pidilizumab, cemiplimab, sintilimab, cetrelimab, spartalizumab, camrelizumab, tislelizumab, balstilimab, toripalimab, dostarlimab, ABBV-181, penpulimab, pembrolizumab, genolimzumab, retifanlimab, sasanlimab, AMP-224, AB122, F-520, MEDI-3387, MEDI-5771, MEDI-0680, SG-001, nivolumab, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01. In some embodiments, the PD-1 antibody is toripalimab.

Any suitable PD-L1 antibody can be used in the present application. Exemplary PD-L1 antibody can be used in the method of the present application includes but is not limited to polyclonal antibody, monoclonal antibody, Fab, scFv, diabody, triabody, minibody, VHH and sdAb. Exemplary PD-L1 antibody includes but is not limited to Manclimab, atezolizumab, avelumab, Cosibelimab, durvalumab, Envafolimab, Socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003.

### Combination Treatment

As used herein, the term “combination treatment,” “combo therapy.” “combo treatment” or “combinational therapy” can be used interchangeably and refer to the administration of a CD73 antagonist and a PD-1/PD-L1 antagonist as described herein to a subject, concurrently or separately.

In some embodiments, the combination treatment comprises a CD73 antagonist and a PD-1/PD-L1 antagonist as described herein. In some embodiments of the combination treatment, the CD73 antagonist and the PD-1/PD-L1 antagonist are administrated to the subject intravenously.

In some embodiments of the combination treatment, the CD73 antagonist is a CD73 antibody as described herein. In some embodiments, the CD73 antibody is administrated to the subject at a dose of about 2 to about 30 mg/kg body weight. In some embodiments, the CD73 antibody is administrated to the subject at a dose of about 2 to about 20 mg/kg body weight. In some embodiments, the CD73 antibody is administrated to the subject at a dose of about 5 to about 20 mg/kg body weight. In some embodiments, the CD73 antibody is administrated to the subject at a dose of about 10 to about 20 mg/kg body weight. In some embodiments, the CD73 antibody is administrated to the subject at a dose of about 15 to about 20 mg/kg body weight. In some embodiments, the CD73 antibody is administrated to the subject at a dose of about 2 to about 15 mg/kg body weight. In some embodiments, the CD73 antibody is administrated to the subject at a dose of about 5 to about 15 mg/kg body weight. In some embodiments, the CD73 antibody is administrated to the subject at a dose of about 10 to about 15 mg/kg body weight. In some embodiments, the CD73 antibody is administrated to the subject at a dose of about 2 to about 10 mg/kg body weight. In some embodiments, the CD73 antibody is administrated to the subject at a dose of about 5 to about 10 mg/kg body weight. In some embodiments, the CD73 antibody is administrated to the subject at a dose of about 2 to about 5 mg/kg body weight. In some embodiments, the CD73 antibody is administrated to the subject at a dose of about 20 to about 30 mg/kg body weight. In some embodiments, the CD73 antibody is administrated to the subject at a dose of about 2, about 5, about 10, about 15 or about 20 mg/kg body weight. In some embodiments, the CD73 antibody is administrated to the subject at a dose of about 20 mg/kg body weight. In some embodiments, the CD73 antibody is administrated to the subject at a dose of about 30 mg/kg body weight.

In some embodiments of the combination treatment, the CD73 antibody is administered to the subject once weekly (QW), once every two weeks (Q2W), once every three weeks (Q3W), once every four weeks (Q4W) or once monthly. In some embodiments, the CD73 antibody is administrated to the subject once every three weeks (Q3W). In some embodiments, the CD73 antibody is administrated to the subject at a dosage of 20 mg/kg once every three weeks (Q3W). In some embodiments, the CD73 antibody is administrated to the subject at a dosage of 30 mg/kg once every three weeks (Q3W).

In some embodiments of the combination treatment, the CD73 antibody includes but is not limited to uliledlimab (I-Mab Biopharma), oleclumab (AstraZeneca), CPI-006 (Corvus Pharma), BMS-986179 (Bristol-Myers Squibb), AB-680 (Arcus Biosciences), NZV-930 (SRF373, Surface Oncolohgy/Novartis), JAB-BX102 (Jacobio), AK119 (Akesobio), Sym024 (Symphogen), IBI 325 (Innovent), BR 101 (Hisun BioRay), and LY3475070 (Eli-Lilly). In some embodiments, the CD73 antibody is uliledlimab (I-Mab Biopharma) or oleclumab (AstraZeneca).

In some embodiments of the combination treatment, the PD-1/PD-L1 antagonist is a PD-1 antibody or a PD-L1 antibody as described herein. In some embodiments of the combination treatment, the PD-L1 antibody includes but is not limited to manelimab, atezolizumab, avelumab, Cosibelimab, durvalumab, Envafolimab, Socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003. In some embodiments, the PD-L1 antibody is atezolizumab.

In some embodiments of the combination treatment, the PD-1 antibody includes but is not limited to pidilizumab, cemiplimab, sintilimab, cetrelimab, spartalizumab, camrelizumab, tislelizumab, balstilimab, toripalimab, dostarlimab, ABBV-181, penpulimab, pembrolizumab, genolimzumab, retifanlimab, sasanlimab, AMP-224, AB122, F-520, MEDI-3387, MEDI-5771, MEDI-0680, SG-001, nivolumab, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01. In some embodiments, the PD-1 antibody is pembrolizumab, nivolumab or toripalimab.

In some embodiments, the PD-1 antibody or a PD-L1 antibody is administered to the subject once weekly (QW), once every two weeks (Q2W), once every three weeks (Q3W), once every four weeks (Q4W) or once monthly. In some embodiments, the PD-1 antibody or a PD-L1 antibody is administrated to the subject once every three weeks (Q3W).

In some embodiments, the combination treatment comprises administrating to the subject uliledlimab at a dosage of 20 mg/kg once every three weeks (Q3W) and Toripalimab at a dosage of 240 mg flat dose once every three weeks (Q3W), intravenously. In some embodiments, the combination treatment comprises administrating to the subject oleclumab at a dosage of 20 mg/kg once every three weeks (Q3W) and Toripalimab at a dosage of 240 mg flat dose once every three weeks (Q3W), intravenously.

In some embodiments, the combination treatment comprises administrating to the subject uliledlimab at a dosage of 30 mg/kg once every three weeks (Q3W) and Toripalimab at a dosage of 240 mg flat dose once every three weeks (Q3W), intravenously. In some embodiments, the combination treatment comprises administrating to the subject oleclumab at a dosage of 30 mg/kg once every three weeks (Q3W) and Toripalimab at a dosage of 240 mg flat dose once every three weeks (Q3W), intravenously.

In some embodiments, the combination treatment comprises administrating to the subject uliledlimab at a dosage of 20 mg/kg once every three weeks (Q3W) and atezolizumab at a dosage of 1200 mg flat dose once every three weeks (Q3W), intravenously. In some embodiments, the combination treatment comprises administrating to the subject oleclumab at a dosage of 20 mg/kg once every three weeks (Q3W) and atezolizumab at a dosage of 1200 mg flat dose once every three weeks (Q3W), intravenously.

In some embodiments, the combination treatment comprises administrating to the subject uliledlimab at a dosage of 30 mg/kg once every three weeks (Q3W) and atezolizumab at a dosage of 1200 mg flat dose once every three weeks (Q3W), intravenously. In some embodiments, the combination treatment comprises administrating to the subject oleclumab at a dosage of 30 mg/kg once every three weeks (Q3W) and atezolizumab at a dosage of 1200 mg flat dose once every three weeks (Q3W), intravenously.

In some embodiments, the combination treatment further comprises a chemotherapy. Exemplary chemotherapy can be used in the combination treatment of the present application includes but is not limited to alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansin and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JIIS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapy include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

### Methods

In one aspect, provided is a method for treating a subject with NSCLC, comprising: (a) determining level of CD73 in a sample of the subject, (b) evaluating whether the subject is more likely to respond to or benefit from a combination treatment of a CD73 antagonist and a PD1/PD-L1 antagonist based on the level of CD73, (c) if the subject is more likely to respond to or benefit from the combination treatment, administering the combination treatment to the subject.

In another aspect, provided is a method for selecting a therapy for a subject with NSCLC, the method comprising: (a) determining level of CD73 in a sample of the subject, and (b) evaluating whether the subject is more likely to respond to or benefit from a combination treatment of a CD73 antagonist and a PD1/PD-L1 antagonist based on the level of CD73, (c) if the subject is more likely to respond to or benefit from the combination treatment, selecting the combination treatment for the subject.

In another aspect, provided herein is a method for identifying a subject with NSCLC is more likely to respond to or benefit from a combination treatment of a CD73 antagonist and a PD1/PD-L1 antagonist, comprising: (a) determining level of CD73 in a sample of the subject, (b) evaluating whether the subject is more likely to respond to or benefit from the combination treatment based on the level of CD73, and (c) if the subject is more likely to respond to or benefit from the combination treatment, providing a recommendation of the combination treatment to the subject.

In another aspect, provided herein is a method for predicting responsiveness of a subject with NSCLC to a combination treatment of a CD73 antagonist and a PD1/PD-L1 antagonist, comprising: (a) determining level of CD73 in a sample of the subject, (b) evaluating whether the subject is more likely to respond to or benefit from the combination treatment based on the level of CD73, and (c) if the subject is more likely to respond to or benefit from the combination treatment, providing a recommendation of the combination treatment to the subject.

In some embodiments of the method of the present application, the NSCLC is a stage III or stage IV NSCLC. In some embodiments, the NSCLC is a stage IV NSCLC. In some embodiments, the NSCLC is an advanced NSCLC. In some embodiments, the NSCLC is a metastatic NSCLC. In some embodiments, the NSCLC is a recurrent NSCLC. In some embodiments, the NSCLC is a refractory NSCLC. In some embodiments, the NSCLC has a squamous histology or a non-squamous histology.

In some embodiments of the method of the present application, the level of CD73 is protein level of CD73. Any suitable method known in the art for determining or measuring the protein level can be used in the method of the present application. In some embodiments, the method determines CD73 protein level expressed on cell membrane. In some embodiments, the method determines CD73 protein level expressed in cytosol. In some embodiments, the method determines CD73 protein level expressed on cell membrane and in cytosol.

In some embodiments, the protein level is determined by immunohistochemistry (IHC), which measures the amount of CD73 protein in a cancer tissue sample, e.g., a NSCLC tissue sample. In some embodiments, the sample is a formalin fixed and paraffin embedded (FFPE) sample. In some embodiments, the sample is an archival sample. In some embodiments, the sample is a fresh sample. In some embodiments, the sample is a frozen sample. In some embodiments, the sample is obtained prior to the combination treatment as described herein.

In some embodiments of the method of the present application, in the IHC test, according to the staining intensity, the level of CD73 protein in each cell (including cell membrane and cytosol) can be assigned with an intensity of 0, 1, 2 or 3, based on the criteria as shown in Table 1.

TABLE 1
CD73 staining Criteria

| Staining intensity | Criteria |
|---|---|
| 0 | no staining |
| 1 | faint or week staining |
| 2 | moderate or mediate staining |
| 3 | intense or strong staining |

The intensity grading can be done manually by an experienced pathologist with conventionally established criteria. Alternatively, various automated/standardized intensity grading systems have been developed. For instance, a quantitative microimmunochemistry assay was published in Kashyap et al., “Quantitative microimmunohistochemistry for the grading of immunostains on tumour tissues,” Nature Biomedical Engineering, volume 3, pages 478-490 (2019). The quantitative assay enables the acquisition of dynamic information, via a metric of the evolution of the immunohistochemistry signal during tissue staining, for the quantification of relative antigen density on tissue surfaces.

In some embodiments, the IHC test determines the percentage of cells with staining intensities of 1, 2 and 3 relative to the total cells. In some embodiments, the IHC test determines the percentage of cells with intensities of 2 and 3 relative to the total cells. In some embodiments, the IHC test determines the percentage of cells with intensity of 3 relative to the total cells.

In some embodiments, the cells include but are not limited to tumor cells, immune cells such as tumor infiltrating immune cells (TILs), stromal cells, vessel cells and any combinations thereof.

In some embodiments, the IHC test determines the tumors with staining relative to the total tumor cells. In some embodiments, the IHC test determines the percentage of tumor cells with staining intensities of 1, 2 and 3 relative to the total tumor cells (TC). In some embodiments, IHC test determines the percentage of tumor cells with staining intensities of 2 and 3 relative to the total tumor cells (TC(+)). In some embodiments, the IHC test determines the percentage of tumor cells with staining intensity of 3 relative to the total tumor cells (TC(++)).

H score captures both the intensity and the proportion of the biomarker from the IHC image and comprises values between 0 and 300, thereby offering a dynamic range to quantify biomarker abundance. In some embodiments of the method of the present application, the IHC test determines the H score of tumor cells (TCH). In some embodiments, the H score of tumor cells refers to the sum of TC, TC(+) and TC(++).

In some embodiments, the IHC test determines the immune cells with staining relative to the total immune cells. In some embodiments, the IHC test determines the percentage of immune cells with staining intensities of 1, 2 and 3 relative to the total immune cells (IC). In some embodiments, IHC test determines the percentage of immune cells with staining intensities of 2 and 3 relative to the total immune cells (IC (+)). In some embodiments, the IHC test determines the percentage of immune cells with staining intensity of 3 relative to the total immune cells (IC(++)).

In some embodiments of the method of the present application, the IHC test determines the H score of immune cells (ICH). In some embodiments, the H score of immune cells refers to the sum of IC, IC(+) and IC(++).

In some embodiments, the IHC test determines the tumor and immune cells with staining relative to the total tumor and immune cells. In some embodiments, the IHC test determines sum of the percentage of tumor cells with staining intensities of 1, 2 and 3 relative to the total tumor cells and the percentage of immune cells with staining intensities of 1, 2 and 3 relative to the total immune cells (TC+IC). In some embodiments, IHC test determines sum of the percentage of tumor cells with staining intensities of 2 and 3 relative to the total tumor cells and the percentage of immune cells with staining intensities of 2 and 3 relative to the total immune cells (TC+IC(+)). In some embodiments, the IHC test determines sum of the percentage of tumor cells with staining intensity of 3 relative to the total tumor cells and the percentage of immune cells with staining intensity of 3 relative to the total immune cells (TC+IC(++)).

In some embodiments, the IHC test determines the tumor or immune cells with staining relative to the total tumor or immune cells. In some embodiments, the IHC test determines the higher one of the percentage of tumor cells with staining intensities of 1, 2 and 3 relative to the total tumor cells and the percentage of immune cells with staining intensities of 1, 2 and 3 relative to the total immune cells (TC/IC). In some embodiments, the IHC test determines the higher one of the percentage of tumor cells with staining intensities of 2 and 3 relative to the total tumor cells and the percentage of immune cells with staining intensities of 2 and 3 relative to the total immune cells (TC/IC(+)). In some embodiments, the IHC test determines the higher one of the percentage of tumor cells with staining intensity of 3 relative to the total tumor cells and the percentage of immune cells with staining intensity of 3 relative to the total immune cells (TC/IC(++)).

In some embodiments of the method of the present application, the subject who is more likely to respond to or benefit from the combination treatment as described herein has a TC of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In some embodiments of the method of the present application, the subject who is more likely to respond to or benefit from the combination treatment as described herein has a TC(+) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In some embodiments of the method of the present application, the subject who is more likely to respond to or benefit from the combination treatment as described herein has a TC(++) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In some embodiments of the method of the present application, the subject who is more likely to respond to or benefit from the combination treatment as described herein has a H score of tumor cells (TCH) of at least 1, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240 at least 250, at least 260, at least 270, at least 280, at least 290, or 300.

In some embodiments of the method of the present application, the subject who is more likely to respond to or benefit from the combination treatment as described herein has a IC of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In some embodiments of the method of the present application, the subject who is more likely to respond to or benefit from the combination treatment as described herein has a IC(+) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In some embodiments of the method of the present application, the subject who is more likely to respond to or benefit from the combination treatment as described herein has a IC(++) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In some embodiments of the method of the present application, the subject who is more likely to respond to or benefit from the combination treatment as described herein has a H score of immune cells (ICH) of at least 1, at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240 at least 250, at least 260, at least 270, at least 280, at least 290, or 300.

In some embodiments of the method of the present application, the subject who is more likely to respond to or benefit from the combination treatment as described herein has a TC+IC of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, at least 110% %, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, or 200% in his/her sample as measured in IHC test as described herein.

In some embodiments of the method of the present application, the subject who is more likely to respond to or benefit from the combination treatment as described herein has a TC+IC(+) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, at least 110% %, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, or 200% in his/her sample as measured in IHC test as described herein.

In some embodiments of the method of the present application, the subject who is more likely to respond to or benefit from the combination treatment as described herein has a TC+IC(++) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, at least 110% %, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, or 200% in his/her sample as measured in IHC test as described herein.

In some embodiments of the method of the present application, the subject who is more likely to respond to or benefit from the combination treatment as described herein has a TC/IC of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

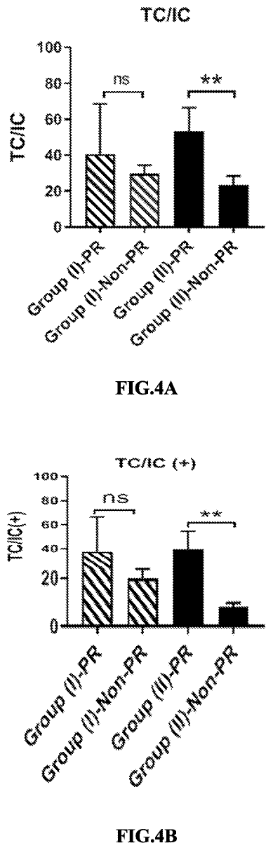

As shown in FIG. 4A, non-responders had a median TC/IC of about 20% while responders had a median TC/IC of about 50%. A TC/IC score that is 25% or higher, therefore, indicates that a corresponding subject is likely to respond to or benefit from the combination treatment. In some embodiments, a TC/IC score that is 30%, 35%, 40%, 45%, 50% or 55%, or higher, therefore, indicates that a corresponding subject is likely to respond to or benefit from the combination treatment.

In some embodiments of the method of the present application, the subject who is more likely to respond to or benefit from the combination treatment as described herein has a TC/IC(+) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

As shown in FIG. 4B, non-responders had a median TC/IC(+) of about 10% while responders had a median TC/IC(+) of about 40%. A TC/IC(+) score that is 15% or higher, therefore, indicates that a corresponding subject is likely to respond to or benefit from the combination treatment. In some embodiments, a TC/IC(+) score that is 20%, 25%, 30%, 35%, 40% or 45%, or higher, therefore, indicates that a corresponding subject is likely to respond to or benefit from the combination treatment.

In some embodiments of the method of the present application, the subject who is more likely to respond to or benefit from the combination treatment as described herein has a TC/IC(++) of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in his/her sample as measured in IHC test as described herein.

In another aspect, provided herein is a method of for identifying a subject with NSCLC is more likely to respond to or benefit from a combination treatment of a CD73 antagonist and a PD1/PD-L1 antagonist, comprising: (a) determining level of CD73 in a sample of the subject, wherein an increase of CD73 in the sample relative to that in a reference sample indicates that the subject is more likely to respond to or benefit from the combination treatment, and (b) providing a recommendation that the subject will be more likely to respond to or benefit from the combination treatment.

In another aspect, provided is a method for predicting responsiveness of a subject with NSCLC to a combination treatment of a CD73 antagonist and a PD1/PD-L1 antagonist, comprising: (a) determining level of CD73 in a sample of the subject, wherein an increase of CD73 in the sample relative to that in a reference sample indicates that the subject is more likely to respond to or benefit from the combination treatment, and (b) providing a recommendation that the subject will have an increased likelihood of being responsive to or benefit from the combination treatment.

In some embodiments, the reference sample comprises a referenced tissue or a reference cell. In some embodiments, the reference sample is derived from a healthy subject, a subject who has experienced first treatment, or a non-diseased sample of the subject.

In another aspect, provided herein is method for treating a subject with non-small cell lung cancer (NSCLC), comprising: (a) identifying a subject of NSCLC as likely to respond to or benefit from a combination treatment of a CD73 antagonist and a PD1/PD-L1 antagonist based on the level of CD73 in sample of the subject, and (b) administering the CD73 antagonist and the PD1/PD-L1 antagonist to the identified subject.

In another aspect, provided herein is a method for treating a subject with NSCLC, comprising administering a CD73 antagonist and a PD1/PD-L1 antagonist to a subject with NSCLC having a level of CD73 in a sample indicating as likely to respond to or benefit from a combination treatment of the CD73 antagonist and the PD1/PD-L1 antagonist.

In another aspect, provided herein is use of a CD73 antagonist and a PD1/PD-L1 antagonist for the manufacture of a medicament for treating a subject with NSCLC having a level of CD73 in a sample indicating as likely to respond to or benefit from a combination treatment of the CD73 antagonist and the PD1/PD-L1 antagonist.

In another aspect, provided herein is use of a CD73 antagonist for the manufacture of a medicament for treating a subject with NSCLC having a level of CD73 in a sample indicating as likely to respond to or benefit from a combination treatment of the CD73 antagonist and a PD1/PD-L1 antagonist, wherein the subject is also administered the PD1/PD-L1 antagonist.

In another aspect, provided herein is use of a PD1/PD-L1 antagonist for the manufacture of a medicament for treating a subject with NSCLC having a level of CD73 in a sample indicating as likely to respond to or benefit from a combination treatment of a CD73 antagonist and the PD1/PD-L1 antagonist, wherein the subject is also administered the CD73 antagonist.

In some embodiments, the increase is determined by TC. In some embodiments, the increase is determined by TC(+). In some embodiments, the increase is determined by H score of tumor cells. In some embodiments, the increase is determined by TC (++). In some embodiments, the increase is determined by IC. In some embodiments, the increase is determined by IC (+). In some embodiments, the increase is determined by IC (++). In some embodiments, the increase is determined by H score of immune cells. In some embodiments, the increase is determined by TC/IC. In some embodiments, the increase is determined by TC/IC (+). In some embodiments, the increase is determined by TC/IC (++). In some embodiments, the increase is determined by TC+IC. In some embodiments, the increase is determined by TC+IC(+). In some embodiments, the increase is determined by TC+IC(++).

In some embodiments of the method of the present application, the subject who is more likely to respond to or benefit from the combination treatment as described herein has an increase of CD73 by at least 10%, 20%, 30%, 40%, 50%, 60%, 70%, 80%, 90%, 100%, 150%, 200%, 250%, 300%, 400%, 500% relative to that in a reference sample.

In some embodiments of the method of the present application, the subject who is more likely to respond to or benefit from the combination treatment as described herein has an increase of TC by at least 10%, 20%, 30%, 40%, 50%, 60%, 70%, 80%, 90%, 100%, 150%, 200%, 250%, 300%, 400%, 500% relative to that in a reference sample. In some embodiments of the method of the present application, the subject who is more likely to respond to or benefit from the combination treatment as described herein has an increase of TC (+) by at least 10%, 20%, 30%, 40%, 50%, 60%, 70%, 80%, 90%, 100%, 150%, 200%, 250%, 300%, 400%, 500% relative to that in a reference sample. In some embodiments of the method of the present application, the subject who is more likely to respond to or benefit from the combination treatment as described herein has an increase of TC (++) by at least 10%, 20%, 30%, 40%, 50%, 60%, 70%, 80%, 90%, 100%, 150%, 200%, 250%, 300%, 400%, 500% relative to that in a reference sample.

In some embodiments of the method of the present application, the subject who is more likely to respond to or benefit from the combination treatment as described herein has an increase of H score of tumor cells by at least 10%, 20%, 30%, 40%, 50%, 60%, 70%, 80%, 90%, 100%, 150%, 200%, 250%, 300%, 400%, 500% relative to that in a reference sample.

In some embodiments of the method of the present application, the subject who is more likely to respond to or benefit from the combination treatment as described herein has an increase of IC by at least 10%, 20%, 30%, 40%, 50%, 60%, 70%, 80%, 90%, 100%, 150%, 200%, 250%, 300%, 400%, 500% relative to that in a reference sample. In some embodiments of the method of the present application, the subject who is more likely to respond to or benefit from the combination treatment as described herein has an increase of IC (+) by at least 10%, 20%, 30%, 40%, 50%, 60%, 70%, 80%, 90%, 100%, 150%, 200%, 250%, 300%, 400%, 500% relative to that in a reference sample. In some embodiments of the method of the present application, the subject who is more likely to respond to or benefit from the combination treatment as described herein has an increase of IC (++) by at least 10%, 20%, 30%, 40%, 50%, 60%, 70%, 80%, 90%, 100%, 150%, 200%, 250%, 300%, 400%, 500% relative to that in a reference sample.

In some embodiments of the method of the present application, the subject who is more likely to respond to or benefit from the combination treatment as described herein has an increase of H score of immune cells by at least 10%, 20%, 30%, 40%, 50%, 60%, 70%, 80%, 90%, 100%, 150%, 200%, 250%, 300%, 400%, 500% relative to that in a reference sample.

In some embodiments of the method of the present application, the subject who is more likely to respond to or benefit from the combination treatment as described herein has an increase of TC/IC by at least 10%, 20%, 30%, 40%, 50%, 60%, 70%, 80%, 90%, 100%, 150%, 200%, 250%, 300%, 400%, 500% relative to that in a reference sample. In some embodiments of the method of the present application, the subject who is more likely to respond to or benefit from the combination treatment as described herein has an increase of TC/IC (+) by at least 10%, 20%, 30%, 40%, 50%, 60%, 70%, 80%, 90%, 100%, 150%, 200%, 250%, 300%, 400%, 500% relative to that in a reference sample. In some embodiments of the method of the present application, the subject who is more likely to respond to or benefit from the combination treatment as described herein has an increase of TC/IC (++) by at least 10%, 20%, 30%, 40%, 50%, 60%, 70%, 80%, 90%, 100%, 150%, 200%, 250%, 300%, 400%, 500% relative to that in a reference sample.

In some embodiments of the method of the present application, the responsiveness comprises a relative increase in one or more of the following: overall survival (OS), progression free survival (PFS), complete response (CR), partial response (PR), stable disease (SD) and combinations thereof. In some embodiments, the responsiveness comprises qualification for a operation such as a surgery.

In some embodiments of the method of the present application, the subject is treatment naïve. In some embodiments, the treatment is first line treatment. In some embodiments, the subject is ineligible or rejected for the first line treatment. In some embodiments, the first line treatment is selected from the group consisting of chemotherapy, PD-1 antibody therapy, PD-L1 antibody therapy and any combination thereof.

In some embodiments, the chemotherapy includes, but is not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustin, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin®) doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitracrine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Ill.), and Taxotere® doxetaxel (Rhone-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further nonlimiting exemplary chemotherapy include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme®) ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

In some embodiments, exemplary PD-1 antibody of the first line treatment includes but is not limited to pembrolizumab, nivolumab, toripalimab, pidilizumab, cemiplimab, sintilimab, cetrelimab, spartalizumab, camrelizumab, tislelizumab, balstilimab, dostarlimab, ABBV-181, penpulimab, genolimzumab, retifanlimab, sasanlimab, AMP-224, AB122, F-520, MEDI-3387, MEDI-5771, MEDI-0680, SG-001, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01.

In some embodiments, exemplary PD-L1 antibody of the first line treatment includes but is not limited to atezolizumab, manelimab, avelumab, cosibelimab, durvalumab, envafolimab, socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003.

In another aspect, provided is a combination of a CD73 antagonist and a PD1/PD-L1 antagonist for use in a treatment of NSCLC in a subject in need thereof, wherein the treatment comprises: (a) determining level of CD73 in a sample of the subject, wherein an increase of CD73 in the sample relative to that in a reference sample indicates that the subject is more likely to respond to or benefit from the combination treatment, and (b) administering the combination treatment to the subject.

### Kit or Article of Manufacture

In another aspect, provided herein is a kit or an article of manufacture for selecting a therapy for a subject with NSCLC, comprising: (1) one or more reagents for determining level of CD73 in a sample from a subject with NSCLC, and (2) a package insert, wherein the package insert suggests treating the subject with a combination treatment of a CD73 antagonist and a PD1/PD-L1 antagonist based on the level of CD73.

In another aspect, provided herein is a kit or an article of manufacture for identifying a subject with NSCLC is more likely to respond to or benefit from a combination treatment of a CD73 antagonist and a PD1/PD-L1 antagonist, comprising (1) one or more reagents for determining level of CD73 in a sample from a subject with NSCLC, and (2) a package insert, wherein the package insert suggests treating the subject with a combination treatment of a CD73 antagonist and a PD1/PD-L1 antagonist based on the level of CD73.

In another aspect, provided herein is a kit or an article of manufacture for use in the method of the present application, comprising (1) one or more reagents for determining level of CD73 in a sample from a subject with NSCLC, and (2) a package insert, wherein the package insert suggests treating the subject with a combination treatment of a CD73 antagonist and a PD1/PD-L1 antagonist based on the level of CD73.

In another aspect, provide herein is a kit or an article of manufacture for use in the method of the present application, comprising: (1) one or more reagents for determining level of CD73 in a sample from a subject with NSCLC, and (2) a package insert, wherein the package insert suggests an increase in the level of CD73 in the sample relative to that in a reference sample indicating the subject is more likely to respond to a combination treatment of a CD73 antagonist and a PD1/PD-L1 antagonist.

In some embodiments, the reagent in the kit or the article of manufacture is an CD73 antibody. Exemplary CD73 antibody can be used in the kit or the article of manufacture includes but is not limited to D7F9A (CST, Cat. No. 13160), 606117 (R&D), EPR6114 (Abcam, Cat. No. ab133582), 4G6E3(Abcam, Cat. No. ab202122), NT5E/2503 (Abcam, Cat. No. ab257309), NT5E/2505 (Abcam, Cat. No. ab257310), NT5E/2545 (Abcam, Cat. No. ab257311), NT5E/2646 (Abcam, Cat. No. ab257312), 7G2 (Thermo Fisher, Cat. No. 1D7), JM11-40 (Thermo Fisher, Cat. No. JM11-40), CD73 Recombinant Rabbit Monoclonal Antibody (1) (Invitrogen, Cat. No. MA5-29454), BLR054F (Thermo Fisher, Cat. No. BLR054F), sc-32299 (Santa Cruz, Cat. No. IE9), and AD2 (Biorad, Cat. No. AD2).

In some embodiments, the kit or the article of manufacture comprises a container and a label or package insert on or associated with the container. Suitable containers include, for example, bottles, vials, syringes, etc. The containers may be formed from a variety of materials such as glass or plastic.

In some embodiments, the kit or the article of manufacture further comprises the CD73 antagonist and the PD1/PD-L1 antagonist of the combination treatment, as described herein.

All publications and patent applications cited in this specification are herein incorporated by reference as if each individual publication or patent application were specifically and individually indicated to be incorporated by reference.

### EXAMPLES

The following examples are put forth so as to provide those of ordinary skill in the art with a complete disclosure and description of how to make and use the present disclosure, and are not intended to limit the scope of what the inventors regard as their disclosure nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Staining of CD73 by IHC

Formalin-fixed paraffin-embedded (FFPE) sections of tumor tissue samples from NSCLC subjects were analyzed through IHC assay by Ventana Benchmark Ultra system. Briefly, FFPE sections were deparaffinized prior to antigen retrieval, blocked and incubated with Abcam Anti-CD73 antibody EPR6114 (Cat. No. ab133582), followed by incubation with an HRP-labeled secondary antibody. Ultra View Universal DAB Detection Kit (Roche 760-500), a substrate of HRP, was used to develop color in positive areas of the tissue.

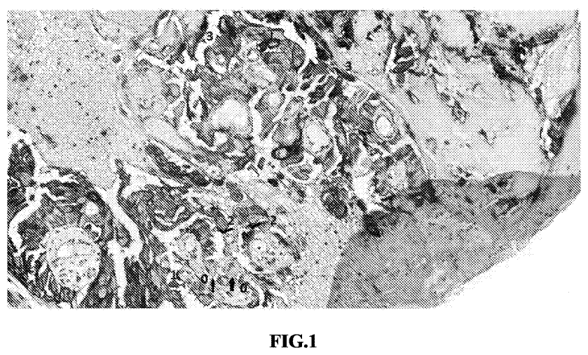

In a tumor sample, CD73 staining intensities of cells existed in dynamic ranges from weak with light brown to strong with dark brown under 4X-20X microscope. Based on the intensities, staining can be classified into no staining (0), faint/weak staining (1), moderate mediate staining (2), and intense/strong staining (3). Exemplary intensities from 0-3 are as shown in FIG. 1.

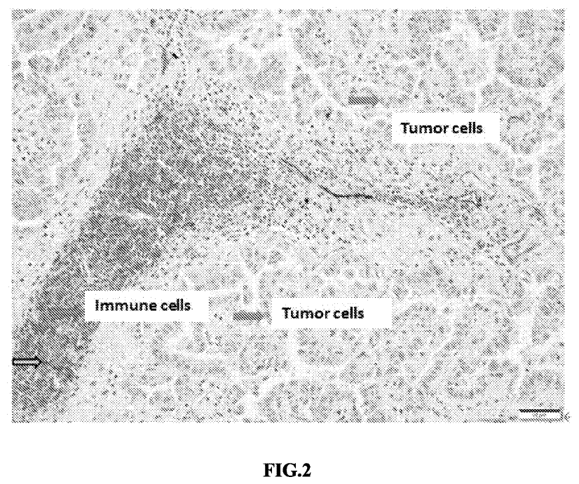

As shown in FIG. 2. The tumor area in the tumor sample normally encompasses tumor cells as well as tumor-associated stroma, including immune cells such as tumor infiltrating immune cells (TILs) which infiltrate immediately adjacent to and contiguous with the main tumor mass.

### Example 2. Scoring of CD73 Level by IHC

To quantify CD73 in tumor samples, novel CD73 IHC scoring systems were established to measure CD73 levels in tumor cells and/or immune cells as shown in Table 2 below.

TABLE 2
CD73 IHC scoring system

| Scoring | |
|---|---|
| system | Description of measurement |
| TC | Percentage of tumor cells staining, with any intensity |
| | (1, 2 and 3) |
| TC (+) | Percentage of tumor cells staining, with moderate or |
| | strong intensity (2 and 3) |
| TC (++) | Percentage of tumor cells staining, with strong |
| | intensity (3) |
| TCH | Tumor cell H score, sum of TC, TC (+) and TC (++) |
| IC | Percentage of immune cells staining, with any intensity |
| | (1, 2 and 3) |
| IC (+) | Percentage of immune cells staining, with moderate or |
| | strong intensity (2 and 3) |
| IC (++) | Percentage of immune cells staining, with strong |
| | intensity (3) |
| ICH | Immune cell H score, sum of IC, IC (+) and IC (++) |
| TC/IC | The higher one of TC and IC |
| TC/IC (+) | The higher one of TC (+) and IC (+) |
| TC/IC (++) | The higher one of TC (++) and IC (++) |
| TC + IC | Sum of TC and IC |
| TC + IC (+) | Sum of TC (+) and IC (+) |
| TC + IC (++) | Sum of TC (++) and IC (++) |

In an on-going phase I/II study of CD73 antibody Uliledlimab, developed by I-Mab Biopharma, at the dose extension stage of the trial, the combination treatment of Uliledlimab and PD-1 antibody Toriparlimab was included as one of the arms. Subjects enrolled in the arm were divided into two subgroups, including:

(I) subjects failed with first line treatment, including SoC chemo and SoC followed by PD-1/PD-L1 antibody treatment, and

(II) subjects naïve from first line treatment, including subjects ineligible for or rejected by SoC chemo.

Prior to the treatment, FFPE sections of tumor samples from the subjects were prepared and subject to IHC staining as described above. The staining results are summarized as below in Table 3.

TABLE 3
IHC staining of CD73

| | | Tumor cells staining | | | Immune cells staining | | |
|---|---|---|---|---|---|---|---|
| | Subject | intensities | | | intensities | | |
| Group | number | 1 | 2 | 3 | 1 | 2 | 3 |
| I | 111201 | 40% | 15% | 20% | 0% | 3% | 0% |
| I | 109201 | 0% | 0% | 0% | 0% | 2% | 0% |
| I | 105209 | N/A | N/A | N/A | 10% | 15% | 0% |
| I | 117204 | 0% | 0% | 95% | 20% | 0% | 0% |
| I | 118214 | 0% | 0% | 0% | 2% | 2% | 0% |
| I | 105205 | 30% | 0% | 0% | 30% | 20% | 0% |
| I | 101201 | 20% | 18% | 50% | 5% | 0% | 0% |
| I | 109204 | 1% | 0% | 0% | 5% | 10% | 0% |
| I | 108201 | 0% | 0% | 0% | 0% | 2% | 0% |
| I | 118210 | 5% | 0% | 0% | 15% | 8% | 2% |
| I | 117203 | 0% | 0% | 0% | 0% | 10% | 0% |
| I | 113203 | 0% | 0% | 0% | 0% | 15% | 0% |
| I | 101202 | 3% | 2% | 90% | 1% | 2% | 2% |
| I | 113202 | 1% | 3% | 2% | 5% | 3% | 0% |
| I | 104205 | 4% | 0% | 0% | 5% | 0% | 0% |
| I | 102201 | 0% | 1% | 0% | 0% | 6% | 0% |
| I | 114205 | 15% | 20% | 15% | 10% | 6% | 0% |
| I | 110202 | 0% | 2% | 1% | 0% | 3% | 0% |
| I | 105208 | 6% | 0% | 0% | 35% | 30% | 0% |
| I | 107203 | 0% | 0% | 0% | 2% | 3% | 0% |
| I | 106202 | 5% | 0% | 0% | 25% | 25% | 20% |
| I | 102202 | 0% | 1% | 2% | 0% | 6% | 4% |
| I | 109205 | 7% | 1% | 0% | 3% | 7% | 0% |
| I | 117201 | 0% | 0% | 0% | 5% | 0% | 0% |
| I | 108202 | 0% | 0% | 0% | 0% | 3% | 0% |
| I | 105211 | 0% | 0% | 0% | 15% | 20% | 5% |
| I | 108204 | 0% | 0% | 0% | 5% | 5% | 0% |
| I | 118207 | 25% | 5% | 0% | 3% | 0% | 0% |
| I | 105210 | 10% | 0% | 0% | 37% | 0% | 0% |
| I | 105206 | 25% | 40% | 15% | 8% | 0% | 0% |
| I | 110204 | 20% | 33% | 45% | 4% | 2% | 0% |
| I | 101204 | 0% | 0% | 0% | 0% | 2% | 0% |
| I | 102203 | 0% | 0% | 0% | 1% | 1% | 0% |
| I | 105203 | 20% | 15% | 0% | 30% | 20% | 0% |
| I | 104206 | 2% | 2% | 0% | 16% | 20% | 4% |
| I | 109207 | 5% | 0% | 0% | 10% | 0% | 0% |
| I | 109206 | 45% | 10% | 25% | 10% | 3% | 2% |
| I | 118206 | 0% | 0% | 0% | 20% | 10% | 5% |
| I | 118202 | 3% | 2% | 0% | 0% | 15% | 70% |
| I | 111202 | 0% | 18% | 80% | 20% | 20% | 5% |
| I | 109203 | 1% | 0% | 0% | 0% | 4% | 2% |
| I | 118211 | 0% | 0% | 0% | 3% | 30% | 2% |
| I | 101205 | 15% | 5% | 3% | 10% | 5% | 5% |
| I | 110203 | 6% | 0% | 0% | 0% | 1% | 0% |
| I | 111203 | 0% | 20% | 60% | 5% | 20% | 0% |
| I | 103204 | 0% | 2% | 0% | 2% | 12% | 1% |
| I | 108203 | 3% | 0% | 0% | 0% | 1% | 0% |
| II | 109202 | 0% | 0% | 0% | 0% | 37% | 3% |
| II | 107202 | 1% | 1% | 97% | 1% | 1% | 0% |
| II | 118224 | 5% | 0% | 0% | 30% | 20% | 0% |
| II | 105201 | N/A | N/A | N/A | 0% | 20% | 0% |
| II | 118217 | 20% | 15% | 5% | 50% | 8% | 0% |
| II | 118204 | 6% | 4% | 0% | 1% | 0% | 0% |
| II | 118229 | 3% | 0% | 0% | 5% | 0% | 0% |
| II | 118218 | 5% | 0% | 0% | 15% | 0% | 0% |
| II | 118225 | 5% | 0% | 0% | 50% | 15% | 5% |
| II | 118230 | 8% | 0% | 0% | 20% | 10% | 0% |
| II | 118216 | 18% | 5% | 0% | 20% | 5% | 0% |
| II | 118231 | 0% | 0% | 0% | 25% | 5% | 10% |
| II | 118203 | 1% | 0% | 0% | 0% | 3% | 0% |
| II | 118221 | 10% | 20% | 0% | 5% | 10% | 0% |
| II | 118228 | 5% | 3% | 0% | 10% | 10% | 5% |
| II | 102204 | 0% | 5% | 0% | 0% | 10% | 0% |
| II | 118212 | 0% | 0% | 0% | 4% | 1% | 0% |
| II | 105202 | 0% | 15% | 0% | 30% | 0% | 0% |
| II | 106203 | 8% | 4% | 0% | 37% | 0% | 0% |
| II | 118227 | 20% | 10% | 0% | 30% | 20% | 0% |
| II | 118215 | 8% | 0% | 0% | 8% | 0% | 0% |
| II | 118226 | 10% | 2% | 0% | 16% | 5% | 0% |

### Example 3. Correlation Between CD73 Level and the Response of the Subjects to the Combination Treatment

The CD73 levels in the samples of the subjects from group (I) and (II) were analyzed prior to the combination treatment. Then subjects of group (I) and (II) were both administrated with Uliledlimab (20 mg/kg, Q3W) and PD-1 antibody Toriparlimab (240 mg, Q3W) for about 42±7 days. After the treatment, the correlation between the response of the subjects to the combination treatment and their CD73 level prior to the treatment was further analyzed, in which PR was defined as at least 30% of tumor regression from the baseline per RECIST v1.1.

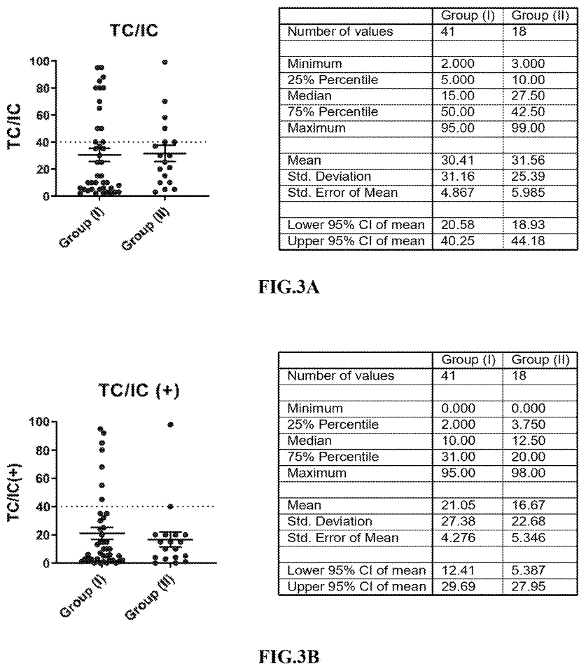

FIGS. 3A and 3B indicate that, prior to the combination treatment, the average CD73 levels in group (I) and (II) were comparable under both TC/IC and TC/IC(+) scoring systems. Among the subjects in group (I), no significant difference in CD73 levels was observed between the responding and non-responding individuals. Surprisingly, subjects with higher CD73 levels in group (II) displayed significantly stronger response to the combination treatment of CD73 antibody and PD-1 antibody, irrespectively under TC/IC (p=0.091) or TC/IC (+) (p=0.0019) scoring system, as shown in FIGS. 4A and 4B.

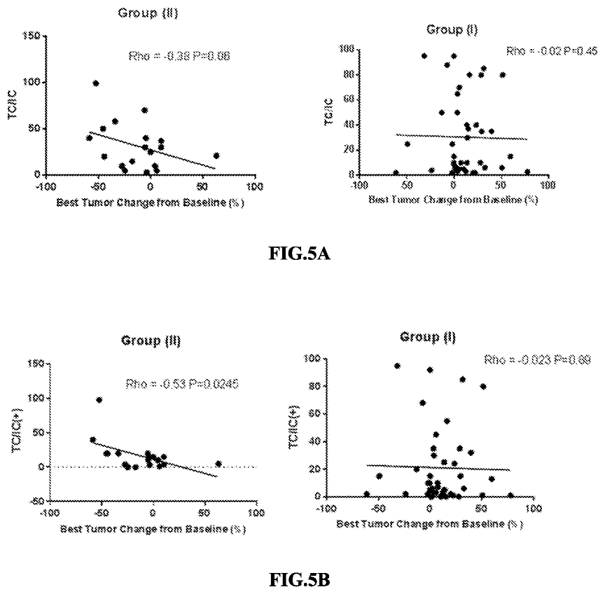

The correlation between CD73 levels and the response to the combination treatment, which was measured as the best tumor change from baseline, in group (I) and (II) were further evaluated via Pearson Correlation Coefficient. As shown in FIGS. 5A and 5B, CD73 levels had stronger correlation with the response to the combination treatment in group (II) as compared to those displayed in group (I), under both TC/IC (rho: −0.38 vs −0.02) and TC/IC (+) (rho: −0.53 vs −0.023) scoring systems, supporting CD73 level as a suitable biomarker for selecting subjects naïve to the first line treatment for the combination treatment of an anti-CD73 antibody and an anti-PD-1 antibody.

### Example 4. Preliminary ROC Evaluation

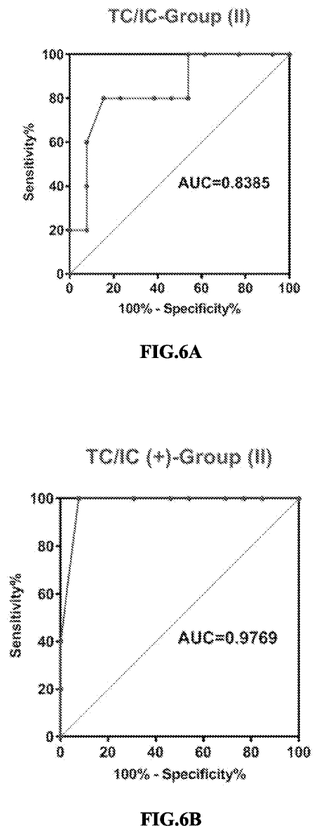

The CD73 level cutoff for selecting subjects naïve to the first line treatment (group II) for the combination treatment can be evaluated through receiver operating characteristic (ROC) analysis. FIGS. 6A and 6B show the preliminary ROC curves under TC/IC and TC/IC (+) scoring systems, respectively. Table 4 below also summarized preliminary results of the TC/IC and TC/IC (+) scoring systems from the ROC curves, indicating either scoring system a reliable approach for subject selection.

TABLE 4
Preliminary summary of the ROC curves

| Scoring system | Area Under the ROC Curve | Youden Index |
|---|---|---|
| TC/IC | 0.8385 | 0.6462 |
| TC/IC (+) | 0.9769 | 0.9231 |

Youden Index = sensitivity + specificity − 1

The present disclosure has been described in terms of particular embodiments found or proposed by the present inventor to comprise preferred modes for the practice of the disclosure. It will be appreciated by those of skill in the art that, in light of the present disclosure, numerous modifications and changes can be made in the particular embodiments exemplified without departing from the intended scope of the disclosure. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

SEQUENCE LISTING

| SEQ | | |
|---|---|---|
| ID NO. | Sequence | Description |
| 1 | EVQLQESGPGLVKPSETLSLTCAVSGYSITSGYYWNWIRQPPGKKLEW | VH of Uliledlimab |
| | MGYINYGGSNGYNPSLKSRITISRDTSKNQFSLKLSSVTAADTAVYYC | |
| | ARDYDAYYEALDDWGQGTTVTVSS | |
| 2 | EIVLSQSPATLSLSPGERATLSCRASSRVNYMHWYQQKPGQSPRPWIS | VL of Uliledlimab |
| | ATSNLASGVPARFSGSGSGTSYTLTISSLEPEDFAVYYCQQWSSNPPT | |
| | FGGGTKVEIK | |
| 3 | SGYYWN | HCDR1 of |
| | | Uliledlimab |
| 4 | YINYGGSNGYNPSLKS | HCDR2 of |
| | | Uliledlimab |
| 5 | DYDAYYEALDD | HCDR3 of |
| | | Uliledlimab |
| 6 | RASSRVNYMH | LCDR1 of |
| | | Uliledlimab |
| 7 | ATSNLAS | LCDR2 of |
| | | Uliledlimab |
| 8 | QQWSSNPPT | LCDR3 of |
| | | Uliledlimab |
| 9 | MCPRAARAPATLLLALGAVLWPAAGAWELTILHTNDVHSRLEQTSEDS | CD73 |
| | SKCVNASRCMGGVARLFTKVQQIRRAEPNVLLLDAGDQYQGTIWFTVY | |
| | KGAEVAHFMNALRYDAMALGNHEFDNGVEGLIEPLLKEAKFPILSANI | |
| | KAKGPLASQISGLYLPYKVLPVGDEVVGIVGYTSKETPFLSNPGTNLV | |
| | FEDEITALQPEVDKLKTLNVNKIIALGHSGFEMDKLIAQKVRGVDVVV | |
| | GGHSNTFLYTGNPPSKEVPAGKYPFIVTSDDGRKVPVVQAYAFGKYLG | |
| | YLKIEFDERGNVISSHGNPILLNSSIPEDPSIKADINKWRIKLDNYST | |
| | QELGKTIVYLDGSSQSCRFRECNMGNLICDAMINNNLRHTDEMFWNHV | |
| | SMCILNGGGIRSPIDERNNGTITWENLAAVLPFGGTFDLVQLKGSTLK | |
| | KAFEHSVHRYGOSTGEFLQVGGIHVVYDLSRKPGDRVVKLDVLCTKCR | |
| | VPSYDPLKMDEVYKVILPNFLANGGDGFQMIKDELLRHDSGDQDINVV | |
| | STYISKMKVIYPAVEGRIKFSTGSHCHGSFSLIFLSLWAVIFVLYQ | |

The invention claimed is:

1. A method for treating a subject with non-small cell lung cancer (NSCLC), comprising:
(a) identifying a subject of NSCLC as likely to respond to or benefit from a combination treatment of a CD73 antagonist and a PD1/PD-L1 antagonist based on the level of CD73 in sample of the subject, and
(b) administering the CD73 antagonist and the PD1/PD-L1 antagonist to the identified subject.

2. A method for selecting a therapy for a subject with NSCLC, the method comprising:
(a) determining the level of CD73 in a sample of the subject, and
(b) evaluating whether the subject is likely to respond to or benefit from a combination treatment of a CD73 antagonist and a PD1/PD-L1 antagonist based on the level of CD73,
(c) selecting the combination treatment for the subject when the subject is determined as likely to respond to or benefit from the combination treatment.

3. A method for treating a subject with NSCLC, comprising administering a CD73 antagonist and a PD1/PD-L1 antagonist to a subject with NSCLC having a level of CD73 in a sample indicating as likely to respond to or benefit from a combination treatment of the CD73 antagonist and the PD1/PD-L1 antagonist.

4. A method for predicting responsiveness of a subject with NSCLC to a combination treatment of a CD73 antagonist and a PD1/PD-L1 antagonist, comprising:
(a) determining the level of CD73 in a sample of the subject,
(b) evaluating whether the subject is likely to respond to or benefit from the combination treatment based on the level of CD73, and
(c) providing a recommendation of the combination treatment to the subject when the subject is determined as likely to respond to or benefit from the combination treatment.

5. A method for identifying a subject with NSCLC as more likely to respond to or benefit from a combination treatment of a CD73 antagonist and a PD1/PD-L1 antagonist than a reference patient, comprising:
(a) determining the level of CD73 in a sample of the subject, wherein an increase of the level of CD73 in the sample relative to that in a reference sample from the reference patient indicates that the subject is more likely to respond to or benefit from the combination treatment than the reference patient, and
(b) providing a recommendation that the subject will be more likely to respond to or benefit from the combination treatment than the reference patient.

6. A method for predicting responsiveness of a subject with NSCLC to a combination treatment of a CD73 antagonist and a PD1/PD-L1 antagonist, comprising:
(a) determining level of CD73 in a sample of the subject, wherein an increase of CD73 in the sample relative to that in a reference sample from a reference patient indicates that the subject is more likely to respond to or benefit from the combination treatment than the reference patient, and
(b) providing a recommendation that the subject will have an increased likelihood of being responsive to or benefit from the combination treatment as compared to the reference patient.

7. The method of any one of claims 1-6, wherein the CD73 antagonist of the combination treatment is a CD73 antibody.

8. The method of claim 7, wherein the CD73 antibody is selected from the group consisting of polyclonal antibody, monoclonal antibody, Fab, scFv, diabody, triabody, minibody, VHH and sdAb.

9. The method of claim 7 or 8, wherein the CD73 antibody comprises:
(a) a HCDR1, a HCDR2, and a HCDR3 within a heavy variable region (VH) having the sequence set forth in SEQ ID No: 1, and
(b) a LCDR1, a LCDR2, and a LCDR3 within a light variable region (VL) having the sequence set forth in SEQ ID No: 2,
wherein the HCDR1, HCDR2, HCDR3, LCDR1, LCDR2, and LCDR3 are according to Kabat numbering scheme.

10. The method of any one of claims 7-9, wherein the CD73 antibody comprises:
(1) a HCDR1 comprising an amino acid sequence as set forth in SEQ ID No. 3,
(2) a HCDR2 comprising an amino acid sequence as set forth in SEQ ID No. 4,
(3) a HCDR3 comprising an amino acid sequence as set forth in SEQ ID No. 5,
(4) a LCDR1 comprising an amino acid sequence as set forth in SEQ ID No. 6,
(5) a LCDR2 comprising an amino acid sequence as set forth in SEQ ID No. 7, and
(6) a LCDR3 comprising an amino acid sequence as set forth in SEQ ID No. 8.

11. The method of any one of claims 7-10, wherein the CD73 antibody is selected from the group consisting of uliledlimab (I-Mab Biopharma), oleclumab (AstraZeneca), CPI-006 (Corvus Pharma), BMS-986179 (Bristol-Myers Squibb), AB-680 (Arcus Biosciences), NZV-930 (SRF373, Surface Oncolohgy/Novartis), JAB-BX102 (Jacobio), AK119 (Akesobio), Sym024 (Symphogen), IBI 325 (Innovent), BR 101 (Hisun BioRay), and LY3475070 (Eli-Lilly).

12. The method of any one of claims 1-11, wherein the PD-1/PD-L1 antagonist of the combination treatment is a PD-1 antibody or a PD-L1 antibody.

13. The method of claim 12, wherein the PD-1 antibody is selected from the group consisting of pembrolizumab, nivolumab, toripalimab, pidilizumab, cemiplimab, sintilimab, cetrelimab, spartalizumab, camrelizumab, tislelizumab, balstilimab, dostarlimab, ABBV-181, penpulimab, genolimzumab, retifanlimab, sasanlimab, AMP-224, AB122, F-520, MEDI-3387, MEDI-5771, MEDI-0680, SG-001, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01.

14. The method of claim 12, wherein the PD-L1 antibody is selected from the group consisting of atezolizumab, manelimab, avelumab, cosibelimab, durvalumab, envafolimab, socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003.

15. The method of any one of claims 1-14, wherein the combination treatment further comprises a chemotherapy.

16. The method of claim 15, wherein the chemotherapy is selected from the group consisting of platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine, and any combination thereof.

17. The method of any one of claims 1-16, wherein the combination treatment comprises administrating the CD73 antagonist and the PD-1/PD-L1 antagonist concurrently or separately.

18. The method of any one of claims 1-17, wherein the NSCLC is a stage III or stage IV NSCLC.

19. The method of any one of claims 1-18, wherein the NSCLC is an advanced NSCLC or a metastatic NSCLC.

20. The method of any one of claims 1-19, wherein the NSCLC is a recurrent NSCLC.

21. The method of any one of claims 1-20, wherein the NSCLC has a squamous histology or a non-squamous histology.

22. The method of any one of claims 1-21, wherein the subject is treatment naïve.

23. The method of claim 22, wherein the treatment is first line treatment.

24. The method of claim 23, wherein the subject is ineligible or rejected for the first line treatment.

25. The method of claim 23 or 24, wherein the first line treatment is selected from the group consisting of chemotherapy, PD-1 antibody therapy, PD-L1 antibody therapy and any combination thereof.

26. The method of claim 25, wherein the chemotherapy is selected from the group consisting of platinum agents (e.g., cisplatin, carboplatin), taxanes agents (e.g., paclitaxel, albumin-bound paclitaxel, docetaxel), vinorelbine, vinblastine, etoposide, pemetrexed and gemcitabine, and any combination thereof.

27. The method of claim 25, wherein the PD-1 antibody is selected from the group consisting of pembrolizumab, nivolumab, toripalimab, pidilizumab, cemiplimab, sintilimab, cetrelimab, spartalizumab, camrelizumab, tislelizumab, balstilimab, dostarlimab, ABBV-181, penpulimab, genolimzumab, retifanlimab, sasanlimab, AMP-224, AB122, F-520, MEDI-3387, MEDI-5771, MEDI-0680, SG-001, BCD-100, BAT-1306, BI-754091, CBT-501, GLS-010, LZM-009, Sym-021, CS-1003, HLX-10, AK-103, AM-0001, ENUM-244C8, ENUM-388D4, JTX-4014, RXI-762, STI-A1110, HLX-20, SSI-361, APL-501, TJ0141H, and SNA-01.

28. The method of claim 25, wherein the PD-L1 antibody is selected from the group consisting of atezolizumab, manelimab, avelumab, cosibelimab, durvalumab, envafolimab, socazolimab, BGB-A333, CK-301, CS-1001, FAZ-053, APL-502, MDX-1105, IMC-001, KD-005, Gensci-047, LY-3300054, SHR-1316, MSB-2311, AVA-004, CBT-502, JS-003, B12 and KY-1003.

29. The method of any one of claims 1-28, wherein the level of CD73 is protein level of CD73.

30. The method of claim 29, wherein the protein level is determined by immunohistochemistry (IHC).

31. The method of any one of claims 1-30, wherein the sample comprises cells selected from the group consisting of tumor cells, immune cells such as tumor infiltrating immune cells, stromal cells and any combinations thereof.

32. The method of any one of claims 1-31, wherein the sample is a formalin fixed and paraffin embedded (FFPE) sample, an archival sample, a fresh sample or a frozen sample.

33. The method of any one of claims 1-32, wherein the sample is obtained prior to the combination treatment.

34. The method of any one of claims 1-33, wherein the reference sample comprises a referenced tissue or a reference cell.

35. The method of claim 34, wherein the reference sample is derived from a healthy subject or a non-diseased sample of the subject.

36. The method of any one of claims 1-35, wherein the responsiveness comprises a relative increase in one or more of the following: overall survival (OS), progression free survival (PFS), complete response (CR), partial response (PR) and combinations thereof.

37. The method of any one of claims 1-36, wherein the level of CD73 comprises a proportion of CD73-positive cells among all cells in the sample.

38. The method of claim 37, wherein the level of CD73 comprises a proportion of CD73-positive tumor cells among all tumor cells in the sample, or a proportion of CD73-positive immune cells among all immune cells in the sample, whichever is higher.

39. The method of claim 38, wherein a CD73 level of 30% or higher identifies the subject as likely to respond to or benefit from a combination treatment.

40. The method of claim 38, wherein a CD73 level of 35%, 40%, 45% or 50% or higher identifies the subject as likely to respond to or benefit from a combination treatment.

41. The method of claim 37, wherein the level of CD73 comprises a proportion of CD73-positive tumor cells among all tumor cells in the sample, or a proportion of CD73-positive immune cells among all immune cells in the sample, whichever is higher, wherein the positive is at least moderate positive.

42. The method of claim 41, wherein a CD73 level of 10% or higher identifies the subject as likely to respond to or benefit from a combination treatment.

43. The method of claim 41, wherein a CD73 level of 15%, 20%, 25%, 30%, 35% or 40% or higher identifies the subject as likely to respond to or benefit from a combination treatment.

44. A kit or an article of manufacture for use in the method of any one of claims 1-43, comprising:
(1) one or more reagents for determining the level of CD73 in a sample from a subject with NSCLC, and
(2) a package insert, wherein the package insert suggests treating the subject with a CD73 antagonist and a PD1/PD-L1 antagonist based on the level of CD73.

45. A kit or an article of manufacture for use in the method of any one of claims 1-43, comprising:
(1) one or more reagents for determining the level of CD73 in a sample from a subject with NSCLC, and
(2) a package insert, wherein the package insert suggests an increase in the level of CD73 in the sample relative to that in a reference sample indicating the subject is more likely to respond to or benefit from a combination treatment of a CD73 antagonist and a PD1/PD-L1 antagonist.

46. The kit or an article of manufacture of claim 44 or 45, wherein the reagent is a CD73 antibody.

47. The kit or an article of manufacture of claim 46, wherein the CD73 antibody is selected from the group consisting of D7F9A (CST, Cat. No. 13160), 606117 (R&D), EPR6114 (Abcam, Cat. No. ab133582), 4G6E3 (Abcam, Cat. No. ab202122), NT5E/2503 (Abcam, Cat. No. ab257309), NT5E/2505 (Abcam, Cat. No. ab257310), NT5E/2545 (Abcam, Cat. No. ab257311), NT5E/2646 (Abcam, Cat. No. ab257312), 7G2 (Thermo Fisher, Cat. No. 1D7), JM11-40 (Thermo Fisher, Cat. No. JM11-40), CD73 Recombinant Rabbit Monoclonal Antibody (1) (Invitrogen, Cat. No. MA5-29454), BLR054F (Thermo Fisher, Cat. No. BLR054F), sc-32299 (Santa Cruz, Cat. No. IE9), and AD2 (Biorad, Cat. No. AD2).

48. Use of a CD73 antagonist and a PD1/PD-L1 antagonist for the manufacture of a medicament for treating a subject with NSCLC having a level of CD73 in a sample indicating as likely to respond to or benefit from a combination treatment of the CD73 antagonist and the PD1/PD-L1 antagonist.

49. Use of a CD73 antagonist for the manufacture of a medicament for treating a subject with NSCLC having a level of CD73 in a sample indicating as likely to respond to or benefit from a combination treatment of the CD73 antagonist and a PD1/PD-L1 antagonist, wherein the subject is also administered the PD1/PD-L1 antagonist.

50. Use of a PD1/PD-L1 antagonist for the manufacture of a medicament for treating a subject with NSCLC having a level of CD73 in a sample indicating as likely to respond to or benefit from a combination treatment of a CD73 antagonist and the PD1/PD-L1 antagonist, wherein the subject is also administered the CD73 antagonist.
